(19) United States
(12) Patent Application Publication
(10) Pub. No.: US 2023/0204472 A1
(43) Pub. Date: Jun. 29, 2023

(54) METHOD FOR PATHOGENS, MICROORGANISMS, AND PARASITES INACTIVATION

(71) Applicant: New York Blood Center, Inc., New York, NY (US)

(72) Inventors: David R. Tabatadze, Worcester, MA (US); Ivan B. Yanachkov, Shrewsbury, MA (US); Boris V. Zavizion, Brookline, MA (US); Bruce Sachais, Wilmington, DE (US)

(21) Appl. No.: 18/171,062

(22) Filed: Feb. 17, 2023

Related U.S. Application Data
(63) Continuation-in-part of application No. 17/262,790, filed on Jan. 25, 2021.
(60) Provisional application No. 62/711,241, filed on Jul. 27, 2018.

(51) Int. Cl. G01N 1/34; A01N 43/44; A01P 1/00; A01N 1/02; A61L 2/00

(52) U.S. Cl. CPC G01N 1/34; A01N 1/0215; A01N 43/44; A01P 1/00; A61L 2/0088; A61L 2202/22

(57) ABSTRACT

Described herein are methods for inactivation or reduction of pathogens, microorganisms or parasites in a sample, media, composition, utility, device, surface or organism by treatment with an alkylating compound of Structure I, followed by elimination or reduction of the residual compound of Structure I by treatment with a neutralizing agent, which eliminates or reduces the toxicity or other undesirable properties of the alkylating compound having Structure I. The neutralizing agent can be present in a treatment solution or be part of a solid-phase agent, and acts by eliminating the alkylating properties of the alkylating compound of Structure I.

### CROSS REFERENCE TO RELATED APPLICATIONS

This application is a continuation-in-part of U.S. Pat. Application Serial No. 17/262,790, filed Jan. 25, 2021, which is an application under section 371 of International patent application number PCT/US2019/043675, filed Jul. 26, 2019, which claims the benefit of priority to U.S. Pat. Application Serial No. 62/711,241, filed Jul. 27, 2018. The entire contents of each of these applications are hereby incorporated by reference.

### FIELD OF THE INVENTION

The present invention relates to compositions and methods for use in the inactivation or reduction of pathogens, microorganisms, or parasites in blood and blood products derived therefrom, including, but not limited to, synthetic blood substitutes, plasma and plasma extracts, packed blood cells and derivatives thereof, platelet particles, including lyophilized versions thereof, oxygen delivery particles, and modified blood cells. Inactivation or pathogen reduction is achieved using alkylating compounds, followed by the elimination or reduction of the residual alkylating compound and/or its by-products.

### BACKGROUND

Blood-derived products including, but not limited to, whole blood and blood products derived therefrom, synthetic blood substitutes, blood cell fractions, plasma and plasma extracts, packed blood cells and derivatives thereof, platelet particles, including lyophilized versions thereof, oxygen delivery particles, and modified blood cells are essential biologics used daily to treat and prevent a wide range of diseases. However, these products are all derived, either directly or indirectly, from donated whole human blood (“source material”). As a result, the source material may be contaminated with known or unknown pathogens (referred to collectively herein after as “microbial contaminants”). Therefore, it is desirable to inactivate microbial contaminants in a broad range of samples, most preferably in the original source material. Currently, there are no universal microbial contaminant reduction techniques that do not damage the source material and/or leave potentially toxic residues.

Targeting and inactivation of pathogens’ nucleic acids is a universal approach to preventing infectivity and can be applied to all classes of microbial contaminants including viruses, bacteria, fungi, protozoa, and other parasites. Some existing methods utilize intercalators, such as, methylene blue, psoralen derivatives (U.S. Pat. Nos. 6,455,286 and 6,133,460), and riboflavin (U.S. Pat. No. 7,985,588), which selectively bind to the nucleic acids and, when photo-activated, damage exerting broad anti-microbial contaminant activity.

Alkylating compounds can inactivate microbial contaminants without the need for photoactivation. The challenge with this approach is to develop compounds which effectively penetrate the pathogens’ cell walls, membranes, and envelopes, and which possess enough selectivity in order to avoid modification of biologic proteins. Even the most selective representatives of alkylating pathogen inactivators, such as, PEN110 (N-(2-aminoethyl)aziridine) and the alkylating intercalator S₃O₃, have insufficient specificity toward nucleic acids, and can damage the source material or leave toxic residues. This may result in the formation of neo-antigens when such alkylating agents are used for treatment of transfusable blood products (Sobral PM et al., Rev Bras Hematol. Hemoter. 2012; 34(3): 231-235; Conlan MG et al., Blood 2004;104(11):382).

U.S. Pat. No. 10,173,976, the entire contents of which is hereby incorporated by reference for all it teaches regarding microbial contaminant inactivation, describes compositions and compounds having two or more aziridinyl groups, interconnected through polyamine constructs, that have high and selective affinity to nucleic acids, low propensity to modify proteins, and can inactivate with a high selectivity, the nucleic acids (e.g. DNA and/or RNA) of pathogens, prokaryotes, or eukaryotes, n a sample.

A drawback of this, and other alkylating agents that generally target nucleic acids for use as pathogen inactivators, is that the residual alkylating compound (for example, in or on the organism, composition, sample, device, utensil, or utility) can be toxic, and cause harm either immediately after pathogen inactivation, or during subsequent use. This drawback can be addressed by removal of the anti-pathogen agent after the pathogen inactivation, or by its inactivation (quenching), i.e. conversion to less harmful or non-harmful substances.

U.S. Pat. No. 7,293,985, the entire contents of which is hereby incorporated by reference for all it teaches regarding microbial contaminant inactivation, describes the use of thiols, e.g., glutathione, a dipeptide containing a cysteine residue, to quench in vitro a pathogen-inactivating compound comprising a nucleic acids intercalator connected to a mustard type alkylating group, wherein the mustard group is capable of reacting in situ to form an electrophilic group. A disadvantage of this method is that it does not provide for sufficient inactivation of this type of nucleic acid-targeting alkylation agent which results in neo-antigens and autoimmunity side effects when blood treated by this method is infused in humans (Conlan MG et al., Blood 2004;104(11):382).

U.S. Pat. Publication No. 2004/0137419, the entire contents of which is hereby incorporated by reference for all it teaches regarding microbial contaminant inactivation, describes a method for the removing of positively charged microbicidal compounds, and in particular PEN110, N-(2-aminoethyl)aziridine, from treated compositions by using cation exchange resins.

There is a need in the art for improved methods of pathogen inactivation that can be applied across a wide range of fields and applications, and particularly, methods of pathogen inactivation that spare proteins and other materials in the treated sample; and for methods that leave little or no toxic compounds/residues in the treated sample.

### SUMMARY OF THE INVENTION

In some aspects, provided herein are compositions and methods for the inactivation and/or reduction of microbial contaminants in a sample by treatment with an alkylating compound, followed by the elimination or reduction of the residual alkylating compound and/or its by-products. The elimination or reduction of the residual alkylating compound can be performed by treatment with a solid-phase agent, which reacts with, or otherwise sequesters, the alkylating compound, or alternatively by treatment with a solution of a neutralizing compound, which eliminates or reduces the toxicity or other undesirable properties of the alkylating compound, by eliminating its alkylating properties followed, in some instances, by removal of the products of neutralization of the alkylating compound and/or the excess of the neutralizing compounds by means of a solid phase agent that sequester them.

In some embodiments, a method for inactivation or reduction of microbial contaminants in a sample is provided, the method comprising: (i) treatment of the sample with a compound or compounds with Structure I:

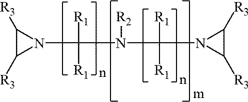

wherein each R₁ is independently selected for each occurrence from H, Cl, F, an alkyl group, CH₃, CH₂CH₃, CH(CH₃)₂, an alkenyl group, a phenyl group, an alkyloxy group, an acyloxy group, or other substituted alkyl group, each R₂ is independently selected for each occurrence from H, an alkyl group, CH₃, CH₂CH₃, CH(CH₃)₂, an alkenyl group, a phenyl group, a cycloalkyl group, an alkyloxy group, or substituted alkyl, alkenyl, cycloalkyl or phenyl group, or a moiety of Structure II:

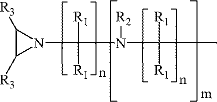

each R3 is independently selected for each occurrence from H, Cl, F, an alkyl group, CH₃, CH₂CH₃, CH(CH₃)₂, an alkenyl group, a phenyl group, an alkyloxy group, an acyloxy group, or other substituted alkyl group; n is independently for each occurrence 3, 4, or 5; m is independently for each occurrence 1, 2, 3, 4, 5, 6, 7, 8, 9 or 10; or a chemically acceptable salt, hydrate, or solvate thereof; and (ii) elimination or reduction of the residual compound(s) having Structure I by treatment with a solid phase agent which reacts with, or otherwise sequesters, the compound, or alternatively by treatment with a solution of a neutralizing compound which eliminates or reduces the toxicity or other undesirable properties of the compound with Structure I, by eliminating its alkylating properties, followed, in some instances, by removal of the products of neutralization of the compound with Structure I and/or the excess of the neutralizing compounds by means of a solid phase agent that sequesters them.

In other embodiments, the neutralization products or their residual by-products can have an undesired effect(s) on the treated sample or render the treated sample, or product containing the sample, unsafe for medicinal use. Therefore, the methods described herein can include removal or reduction of the neutralization agent and/or residual by-products using a solid phase agent which either chemically reacts with, and covalently binds, absorbs, or otherwise sequesters the neutralization products, by-products and/or excess neutralizing compound(s), followed by removal of the solid phase agent. The solid phase agent can be functionalized with thiosulfate groups (—S—SO₃⁻Na⁺) or with epoxy groups, which react with and sequester mercaptan or thiol types of neutralizing compounds. In some embodiments, the solid phase agent can be a cationite or an anionite, which sequester, through an ion-exchange, the cationic type products of neutralization or anionic type of neutralizing compounds. In other embodiments, the solid phase agent can be an absorbing solid phase agent, such as activated carbon that absorbs with high affinity polyamines or a sulfur containing organic moiety.

In some embodiments, after treatment of pathogen-containing samples with compounds of Structure I, the residual compounds are removed by treatment with a solid phase agent that includes reactive groups which react with, and covalently bind, the compound(s) of Structure I, followed by removal of the solid phase agent by filtration or other means. Non-limiting examples of such reactive groups include, thiosulfate, —OS(O)(O⁻)S⁻, thiosufonate, —S(O)(O⁻)S⁻, mercapto or thiol groups, substituted mercapto or thiol groups, thioureas, thiocarboxylic or dithiocarboxylic acids, thiocarbonate or dithiocarbonate O-esters, thiophosphonate, and thiophosphates. The thiol groups can have a pKa of less than 9. In other embodiments, the thiol groups can have a pKa of less less than 8. In some embodiments, the solid phase agent includes not only the reactive groups, but other groups, which without reacting with the compounds of Structure I, enhance their reactivity by protonating them, or non-covalently binding them, increasing their local concentration, or enhancing the reactivity of the reactive groups. In yet other embodiments, the solid phase agent includes non-reactive hydrophilic groups, such as polyethylene glycol, which improve its wettability in aqueous media and reduce its undesired effects on the components of the treated media.

In some embodiments, the method for microbial contaminant inactivation is performed ex vivo on samples that are then returned (for example, transfused) to the animal.

In other embodiments, the method for inactivation of microbial contaminants is performed ex vivo on samples wherein compounds of Structure I are removed ex vivo together with residual neutralization product and by-products.

The microbial contamination and neutralization agent removal methods disclosed herein can be used on many sample types including, but not limited to, whole blood and blood products derived therefrom, synthetic blood substitutes, blood cell fractions, plasma and plasma extracts, packed blood cells and derivatives thereof, platelet particles, including lyophilized versions thereof, oxygen delivery particles, and modified blood cells.

In some embodiments disclosed herein, the compound of Structure I comprises N1,N4-bis(3-(aziridin-1-yl)propyl)-N1,N4-dimethylbutane-1,4-diamine (ZD010) as the alkylating agent and sodium thiosulfate (Na₂S₂O₃) as the neutralization agent. The thiosulfate is added to the ZD010 treated sample after a suitable inactivation period (from five minutes to 24 hours including all time intervals there between) resulting in a viable microbial contaminant-free sample further containing a biologically inert and well tolerated neutralization product (S,S′-(7,12-dimethyl-3,7,12,16-tetraazaoctadecane-1,18-diyl) bis(O-hydrogen sulfothioate)). In some embodiments, a suitable inactivation period can be one hour, four hours, six hours, eight hours or twelve hours. Moreover, the methods disclosed herein further comprise complete extraction of this biologically inert neutralization product from the treated sample using filtration through a suitable chromatography substrate, such as but not limited to, a polystyrene sulfonate (PSS) cartridge in a single closed system.

Further embodiments can include a leukocyte filtration step to remove white blood cells either before or after treatment with the compound of Structure I, and further processing of the leukocyte-free sample.

Sample collection and microbial contaminant inactivation systems are described herein. In some embodiments, a sample collection and microbial contaminant inactivation system comprises: a venipuncture apparatus in fluid contact with a sample collection bag having a buffered anticoagulant diluent therein; a means attached to said sample collection bag for aseptically introducing an alkylating substance into said bag; a second means attached to said sample collection bag for introducing an alkylating substance neutralizing agent into said sample collection bag; and a means attached to said sample collection bag for aseptically removing said sample from said sample collection bag. In some embodiments, the means is an administration means.

The buffered anticoagulant diluent can be citrate dextrose phosphate. The alkylating substance can be ZD010. The alkylating substance neutralizing agent can be sodium thiosulfate. In some embodiments, the system can further comprise a leukocyte reduction apparatus. The sample can be whole blood.

In other embodiments, a sample collection and microbial contaminant inactivation system comprises a venipuncture apparatus in fluid contact with a first sample collection bag having a buffered anticoagulant diluent therein; a means attached to said first sample collection bag for aseptically introducing an alkylating substance into said first sample collection bag; a means for removing said alkylating substance from said sample wherein said means for removing said alkylating substance from said sample is separate from and in fluid contact and downstream from said first sample collection bag; a second sample collection bag aseptically attached to said means for removing said alkylating substance from said sample; a means attached to said second sample collection bag for introducing a residual alkylating substance neutralizing agent into said second sample collection bag; and a means attached to second sample collection bag for aseptically removing said sample from said sample collection bag. In some embodiments, the means is an administration means.

The buffered anticoagulant diluent can be citrate dextrose phosphate. The alkylating substance can be ZD010. The alkylating substance neutralizing agent can be sodium thiosulfate. In some embodiments, the means for removing said alkylating substance from said sample comprises a chromatography substrate housed in a column or other suitable flow through device. In other embodiments, the system can further comprise a leukocyte reduction apparatus. The sample can be whole blood.

In some embodiments, a sample collection and microbial contaminant inactivation system comprises an outer bag having a buffered anticoagulant diluent therein; an inner bag included within said outer bag, wherein said inner bag includes an alkylating substance for introduction into said outer bag. The buffered anticoagulant diluent can be citrate dextrose phosphate. The alkylating substance can be ZD010. The alkylating substance neutralizing agent can be sodium thiosulfate.

In some embodiments, the means for removing said alkylating substance from said sample comprises a chromatography substrate housed in a column or other suitable flow through device. In other embodiments, the system can further comprise a leukocyte reduction apparatus. The sample can be whole blood.

### BRIEF DESCRIPTION OF THE DRAWINGS

FIG. 1 shows the interaction of a compound of Structure I with a solid phase agent having nucleophilic thiol groups attached through a linker L, and in which accessory anionic sulfo-groups are directly attached to the polymer P matrix.

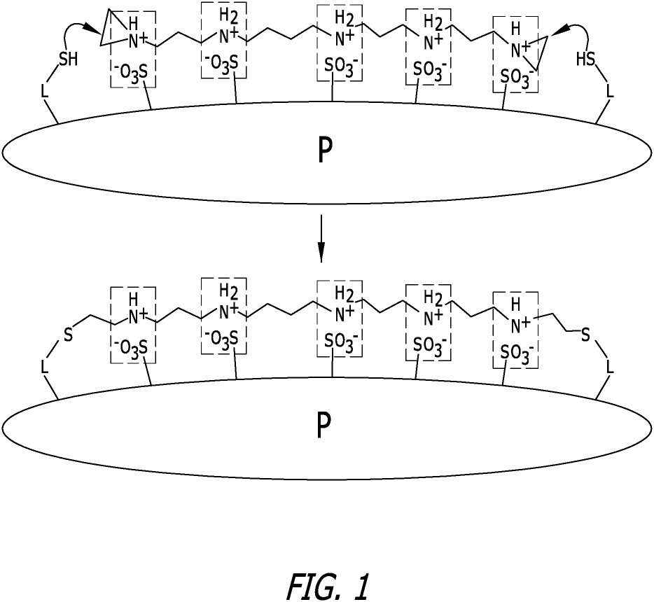

FIG. 2 shows a closed, complete system for treating a freshly collected sample with ZD010, and employing a ZD010 extraction cartridge prior to addition of the thiosulfate quencher.

FIG. 3 shows a closed, complete system for treating a freshly collected sample with ZD010 using a thiosulfate quencher without using an elimination cartridge.

FIG. 4 shows a multi-bag sample collection and processing system comprising a multi-bag closed-system.

### DETAILED DESCRIPTION OF THE INVENTION

The term “sample” as used herein refers to, but is not limited to, whole blood collected using conventional venipuncture techniques, including modifications thereof such as indwelling arterial and venous collection lines and whole blood components collected using apheresis techniques. Sample can also include blood products derived from whole blood, such as but not limited to, synthetic blood substitutes, blood cell fractions, plasma and plasma extracts, packed blood cells and derivatives thereof, platelet particles, including lyophilized versions thereof, oxygen delivery particles and modified blood cells.

The term “oxygen delivery particles” as used herein include, but are not limited to, lipidic oxygen-containing microparticles (LOMs) comprising a lipid shell and an oxygen gas (O2) core, with an approximate diameter of 4 µm. These particles were designed to mix with venous blood and deliver O₂ to oxygen-deprived hemoglobin-the molecule that carries oxygen to all tissues within the body. When administered intravenously to asphyxiated (and therefore hypoxemic) animals, the LOMs are able to maintain full-body oxygenation, normal blood pressure, and normal heart rate.

The terms “neutralizer,” “neutralizer compound” or “neutralizer agent,” when used in the context of compound(s) of Structure I, designate molecules that, in general, can react and open aziridinyl groups of the compounds of Structure I in a sample. In some embodiments, neutralizer can refer to sodium thiosulfate (STS).

The term “ZD010” as used herein refers to a specific embodiment of Structure I comprising N1,N4-bis(3-(aziridin-1-yl)propyl)-N1,N4-dimethylbutane-1,4-diamine.

The term “solid phase agent” used in the context of the methods described herein is defined as a solid that is insoluble in the media of the sample, and that is used to remove the compound of Structure I, or the products of inactivation of compound of Structure I, or the products of chemical transformation, or degradation of the compounds of Structure I or the neutralizing agent from the sample. In one embodiment, the solid phase agent is suitable for the removal of residual ZD010, residual STS and/or STS complexed with (neutralized) ZD010 forming S,S′-(7,12-dimethyl-3,7,12,16-tetraazaoctadecane-1,18-diyl) bis(O-hydrogen sulfothioate).

The term “microbial contaminant” as used herein refers to pathogens, including viruses, bacteria, or any other microorganisms, or eukaryote, single-, or multicellular eukaryote, including, but not limited to fungi, protozoa, single- or multicellular parasite including helminths, schistosomes or nematodes or their eggs, single or multicellular algae and of crustacean, or any other undesirable organisms or infectants.

The invention provides a method for contaminant inactivation/reduction in a sample by treatment with an inactivating compound of Structure I followed by removal or neutralization (quenching) of the residual compound of Structure I:

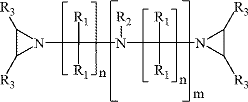

wherein: each R₁ is independently selected for each occurrence from H, CH₃, CH₂CH₃, CH(CH₃)₂, Cl, F, an alkyl group, an alkenyl group, a phenyl group, an alkyloxy group, an acyloxy group, or substituted alkyl group, each R₂ is independently selected for each occurrence from H, CH₃, CH₂CH₃, CH(CH₃)₂, an alkyl group, an alkenyl group, a phenyl group, a cycloalkyl group, an alkyloxy group, or substituted alkyl, substituted alkenyl, substituted cycloalkyl or substituted phenyl group, or a moiety of Structure II:

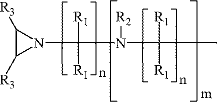

each R₃ is independently selected for each occurrence from H, CH₃, CH₂CH₃, CH(CH₃)₂, Cl, F, an alkyl group, an alkenyl group, a phenyl group, an alkyloxy group, an acyloxy group, or other substituted alkyl group; each n is independently for each occurrence 3, 4, or 5; each m is independently for each occurrence 1, 2, 3, 4, 5, 6, 7, 8, 9 or 10; or a chemically acceptable salt, hydrate, or solvate thereof.

In some embodiments, the inactivating compound of Structure I can include:

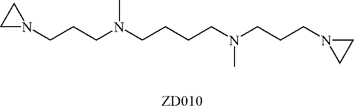

According to one method of the present invention, the microbial contaminants in the sample are treated with compound of ZD010. The compound ZD010 can be formulated as a liquid, solution, gel, solid, powder, particles, or can be encapsulated, dissolved, dispersed, pulverized, micronized, or converted to nano-particles, or in other formulated forms or in combinations thereof. The solvent for the compositions of the compounds of ZD010 can be water, aqueous buffers, or aqueous salt solutions, organic solvents, such as, but not limited to, dimethylsulfoxide, dimethylacetamide, ethanol, isopropanol, acetone, polyethylene glycol(s) of different molecular masses, glycerol, propylene glycol, benzyl alcohol, or mixtures thereof, liquidities gasses, or mixtures thereof. The solvents can include various organic or inorganic additives, stabilizers, activators, or adjuvants.

In some embodiments disclosed herein, the sample containing a microbial contaminant is treated with ZD010 for a period of time from 30 sec to 72 hours, from 20 min to 24 h, or from 60 min to 8 h, and at temperatures from 0 to 100 ℃, from 10 to 60° C., or from 20 to 40° C.; and at pH from 1 to 14, from 4 to 9, or from 6 to 8; and at concentrations from 10 nM to 10 mM, from 1 µM to 1 mM, or from 100 µM to 500 µM.

The microbial contaminant inactivation effect of ZD010 can increase with the increase of their concentration, dose or amount, treatment time, and/or temperature. At the same time, possible undesired effects on the treated sample can also increase with the compound concentration, dose or amount, time and/or temperature of treatment. The user of the method can determine the optimal concentration, dose or amount of ZD010, time and temperature of treatment based on the type and properties of the treated media and the nature and type of pathogens or undesired organisms present in it, and the desired level of their inactivation. For example, the optimal treatment temperature for a sensitive sample, such as for instance, platelets concentrate can be room temperature, and the treatment time can be restricted to 1-2 h or less, while for more heat tolerant samples, such as heat-treated animal sera, the optimal temperature can be 40° C. or more, at a treatment time of 1-6 h. The user can determine the optimal concentration, dose, or amount of ZD010, and the time and temperature of treatment by experimentation, using the approaches disclosed herein, and similar approaches known to a person of ordinary skill in the art.

The optimal treatment parameters (concentration, time, temperature) can depend not only on the properties of the treated sample and the type and nature of the pathogens or other undesired organisms present in it, but also on the desired degree of their inactivation/reduction, which can depend on the intended use of the treated sample. For example, if the treated sample is whole human blood intended for use as a transfusion product or biologic such as, but not limited to, gamma globulin or plasma, a reduction/inactivation level of more than 9 logs can be required to achieve full sample sterilization.

In some embodiments of this invention, the alkylating properties of the compounds of ZD010, and therefore their cytotoxicity resulting from those alkylating properties can be reduced or removed by treatment of the sample, where residual compound of ZD010 is present, with small nucleophilic molecules or ions, such as, but not limited to, thiosulfate, sodium thiosulfate, thiophosphate, sodium thiophosphate and/or others as set forth in U.S. Pat. Publication No. 2021/0227827 (the entire contents of which related to ZD010 neutralizing agents is incorporated herein by reference).

In some embodiments of this invention, thiol ZD010 neutralizers can have a pKa close to the pH of the media in which the inactivation takes place, i.e., if the neutralization takes place at pH 7, or close to pH 7, the thiol type neutralizer can have a pKa close to 7, which can provide a compromise between the increase of the nucleophilicity of the anionic form of the neutralizer with the increase of its basicity and the decrease of the concentration of the anionic form with the increase of its pKa above the pH of the media.

It is understood that the optimal conditions for the fastest and most efficient neutralization of the residual ZD010 in the treated media can be different among various conditions, such as but not limited to, the type of media, and the type of neutralizing compound, and they can be reasonably selected and optimized experimentally by a person with ordinary skill in the art by using the methods disclosed herein or similar experimental methods.

The neutralization, or degree of reduction of the amount of the residual ZD010 can be less than 50%, more than 2 times, more than 10 times, i.e., 1 log, more than 2 logs, at least 3 logs, at least 4 logs, at least 5 logs, at least 6 logs, at least 7 logs, by at least 8 logs, at least 9 logs, at least 10 logs, or more than 10 logs. In other embodiments, the neutralization or degree of reduction of the amount of the residual ZD010 can be 1 log, 2 logs, 3 logs, 4 logs, 5 logs, 6 logs, 7 logs, 8 logs, 9 logs, 10 logs, about 1 log, about 2 logs, about 3 logs, about 4 logs, about 5 logs, about 6 logs, about 7 logs, about 8 logs, about 10 logs, between about 1 to about 3 logs, between about 2 to about 5 logs, between about 3 to about 7 logs, between about 4 to about 8 logs, between about 5 logs to about 9 logs, or between about 5 logs to about 10 logs.

In some embodiments of the invention, the product(s) of neutralization of the compound(s) with ZD010, i.e., the products of their reaction with the neutralizing compound(s), or the products of reaction of compounds with ZD010 with the components of the treated sample can have undesired properties for the intended use. In other cases, the neutralizing compounds can have undesirable properties. In all those cases, the products of neutralization or products of reaction, or the neutralizing compounds can be removed from the treated sample, or their amount can be reduced, by treatment of the sample with a solid phase agent which is insoluble in the treated media, and which solid phase agent chemically reacts with and covalently binds, or absorbs, or otherwise sequesters the products of neutralization or reaction of the ZD010 and/or the neutralizing compound(s). After the treatment, the solid phase agent can be removed from the treated media by filtration, centrifugation, sedimentation or other appropriate physical means. Alternatively, the solid phase agent can be in contact with the treated media through a membrane, pouch or other appropriate physical barrier, which is permeable by the products of neutralization or the products of reaction of the ZD010 with the components of the treated sample, or the neutralizing compound(s) and is not permeable by the solid phase agent.

The solid phase agent can be a porous organic polymer of micro-, or macroporous, or gel type, or it can be any highly porous solid of organic or inorganic type, such as but not limited to, amorphous carbon, activated carbon, charcoal, silica gel, titania, circonia, or it can be a non-porous solid with high dispersity, i.e., of small particle size that provides for high surface to volume ratio. The solid phase agent can also be of mixed type, for example, solid non-porous particles, which are covered with a layer of porous material.

In some embodiments, an organic polymer can be cross-linked. In other embodiments, the organic polymer can be, but is not limited to, a polystyrene polymer, a polyacrylate polymer, a polymethacrylate polymer, a polyurethane polymer, a polyamide polymer, a dextran polymer, such as, but not limited to Sephadex®, a agarose polymer, such as but not limited to Sepharose®, a cellulose based polymer, a modified cellulose based polymer, such as but not limited to carboxymethylcellulose, diethylaminoethyl cellulose, methylcellulose, other polysaccharide, any other linear, branched, or cross-linked homo- or hetero-polymer or block copolymer, with iso- or atactic configuration, or with other tacticity, or can be any other appropriate macromolecule that is not soluble in the treated media.

The methods of the invention described herein, can be used to inactivate not only pathogenic microorganisms, but also non-pathogenic cells, such as leukocytes, when their presence in the treated sample is not desirable, as for instance in transfusable blood or blood products. The methods provided herein, can be used for the treatment of a sample. The sample can include, but is not limited to, whole blood and blood products derived therefrom, synthetic blood substitutes, blood cell fractions, plasma and plasma extracts, packed blood cells and derivatives thereof, platelet particles, including lyophilized versions thereof, oxygen delivery particles and modified blood cells.

The methods for pathogen inactivation described herein, can be performed in transfusion blood or blood products, in which the treatment with the ZD010 and the following treatment for their removal, inactivation, and products or inactivation and/or inactivators’ removal is done in a sterile system which can be a partially or fully closed system.

In some embodiments, a leukocyte reduction step can be performed prior to or after ZD010 treatment. Methods of leukocyte reduction include centrifugation, washing, freezing, buffy coat removal and/or filtration. Selective leukocyte filters are generally the most efficient and can be used in-line with the blood collection process. Selective filtration technologies combine depth and adsorption filtration to achieve the highest efficiency. Depth filters are usually composed of densely packed fibers to remove particles, either by adherence or absorption onto the fibers, or by entrapment between fibers as particles pass through the filter. Adsorption filters utilize the properties of white blood cells, which selectively adhere to filter fibers. Suitable representative leukocyte filters include, but are not limited to, PLASMAFLEX™ In-line Plasma leukoreduction filter (Macropharma Corporation); IMUGARD™ III, a leukocyte removal filter system, (Terumo BTC); and Sepacell® RZ-2000 (Asahi Kasei Bioprocess America, Inc.). Each of these leukofiltration (leukoreduction) systems should be used in accordance with the manufacturer’s instructions as set forth in their respective U.S. Food and Drug Administration (USFDA) approved instructions for use. The embodiments set forth herein can be used for sample collection or fractions thereof from a donor. Whole blood is generally collected using standard venipuncture techniques known to those of ordinary skilled in the art. Alternatively, selected blood components, such as but not limited to, plasma (plasmapheresis), platelets (plateletpheresis) or leukocytes (leukapheresis) can be collected from donors using apheresis. The process of apheresis involves removal of whole blood from a donor with an instrument that is essentially designed as a centrifuge wherein whole blood components are separated. One of the separated portions is then withdrawn and the remaining components may be transfused back into the donor. Whole blood is introduced into a chamber that is spinning, and the blood separates into components (e.g., plasma; platelet rich plasma; leukocytes; red blood cells) by gravity along the wall of the chamber. The desired component to be removed can be selected by moving the level of an aspiration device.

In donor apheresis, a healthy person donates blood using an apheresis machine, which is programmed to collect the desired blood component - either red blood cells, white blood cells, platelets, or plasma. The component can be stored and distributed to hospitals, to be given to a patient in need.

Donated apheresis plasma can be frozen or sent to a processing facility, where it is treated and processed to create pharmaceutical products, such as but not limited to, gamma globulin. Platelets can be donated as often as every seven days. However, with double apheresis red blood cell donations, a donor has to wait up to at least 16 weeks before donating again.

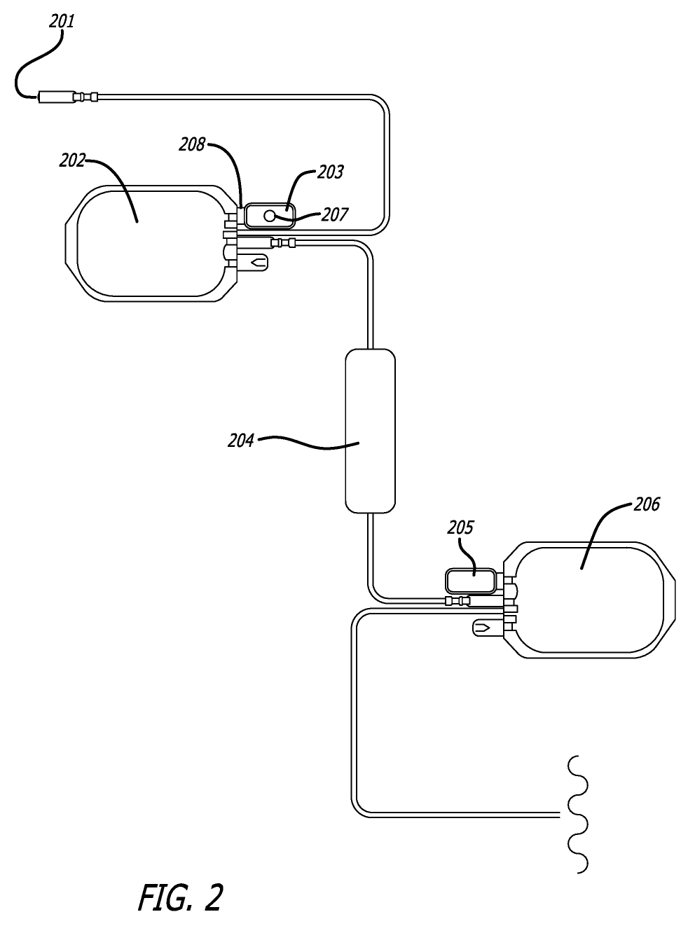

Described herein are sample collection and microbial contaminant inactivation systems. FIG. 2 depicts a sample collection and microbial contaminant inactivation system described herein. The systems can use a venipuncture apparatus to collect blood. In some embodiments, the venipuncture apparatus can be a phlebotomy needle, any type of needle, and/or any type of syringe. As depicted in FIG. 2, the venipuncture apparatus can be phlebotomy needle (201). Blood is collected by phlebotomy needle (201) and flows into collection bag (202). Collection bag (202) can include a buffered anticoagulant diluent. In some embodiments, the anticoagulant diluent is citrate dextrose phosphate (CPD) solution.

Collection bag (202) can be configured to attach to one or more administration means for adding the alkylating agent. As used herein administration means can include, for example but not limited to, a syringe or a pre-filled container attached to the collection bag (permanently or detachably). In some embodiments, the alkylating agent can be, but is not limited to, ZD010. In some embodiments, collection bag (202) can be configured to attach to 1, 2, 3, 4, 5, or 6 administration means, at least one administration means, or more than one administration means. FIG. 2 depicts at least one administration means (203) for adding an alkylating agent, such as but not limited to, ZD010. Administration means (203) as is configured to attach to collection bag (202). Administration means (203) can comprise a five to 30 milliliter container including a buffered anticoagulant diluent. In some embodiments, the buffered anticoagulant diluent is a citrate dextrose phosphate (CPD) solution. Administration means (203) can further comprise capsule (207). In some embodiments, capsule (207) can comprise ZD010. The contents of capsule (207) can remain intact until ruptured/broken. Capsule (207) can be ruptured mechanically through force applied to the exterior of the collection bag directly or indirectly to the capsule thereby releasing its contents, e.g. ZD010, into the CPD solution. After the ZD010 mixes with the CPD solution, the resulting mixture can then be emptied into collection bag (202). The means for opening (208) means (203) can be a stopcock valve, a pinch valve, a valve, a flexible membrane or other suitable means known to those having ordinary skill in the art. Once the resulting mixture (i.e. ZD010 and CPD solution) is added to collection bag (202), the solution/mixture comprising the ZD010 (or other suitable alkylating substance disclosed herein), CPD solution (or other suitable buffered anticoagulant diluent disclosed herein), and the collected blood incubates. In some embodiments, the incubation occurs at room temperature. In other embodiments, the incubation occurs at 15° C., 16° C., 17° C., 18° C., 19° C., 20° C., 21° C., 22° C., 23° C., 24° C., 25° C., about 15° C., about 16° C., about 17° C., about 18° C., about 19° C., about 20° C., about 21° C., about 22° C., about 23° C., about 24° C., about 25° C., between about 15° C. to about 18° C., between about 18° C. to about 21° C., or between about 19° C. to about 25° C.

In yet other embodiments, the incubation occurs at 65° F., 66° F., 67° F., 68° F., 69° F., 70° F., 71° F., 72° F., 73° F., 74° F., 75° F., about 65° F., about 66° F., about 67° F., about 68° F., about 69° F., about 70° F., about 71° F., about 72° F., about 73° F., about 74° F., about 75° F., between about 65° F. to about 68° F., between about 68° F. to about 71° F., or between about 69° F. to about 75° F. Incubation can occur over an incubation period of at least one hour, at least two hours, at least three hours, at least four hours, at least five hours, at least six hours, at least seven hours, at least eight hours, at least nine hours, at least ten hours, 1 hour (hr), 2 hours (hrs), 3 hrs, 4 hrs, 5 hrs, 6 hrs, 7 hrs, 8 hrs, 9 hrs, 10 hrs, about 1 hr, about 2 hrs, about 3 hrs, about 4 hrs, about 5 hrs, about 6 hrs, about 7 hrs, about 8 hrs, about 9 hrs, about 10 hrs, more than 1 hour, more than 2 hrs, more than 3 hrs, more than 4 hrs, more than 5 hrs, more than 6 hrs, more than 7 hrs, more than 8 hrs, more than 9 hrs, more 10 hrs, between about 60 minutes (mins) to about 120 mins, between about 1 hr to about 3 hrs, between about 3 hrs to about 6 hrs, or between about 4 hrs to about 8 hrs.

In one embodiment, once the alkylating substance is added to collection bag (202), the solution/mixture comprising the alkylating substance, the buffered anticoagulant diluent, and the collected blood incubates at a temperature of about 20° C. for about six hours.

As depicted in FIG. 2, the sample collection and microbial inactivation system can further comprise solid phase removal column (204). Solid phase removal column can be positioned up stream of a final treated sample collection bag. FIG. 2 illustrates solid phase removal column (204) upstream of final treated sample collection bag (206). In some embodiments, an additional treatment can be performed using an alkylating substance neutralizing agent, such as but not limited to STS, included in second administration means (205). The alkylating substance neutralizing agent can be added to final treated sample collection bag (206). The alkylating substance neutralizing agent can be sodium thiosulfate. The blood treated with the alkylating substance neutralizing agent can be incubated for an additional period of time at/or about room temperature. In some embodiments, the additional period of time is about 1 hr, about 2 hrs, about 3 hrs, about 4 hrs, about 5 hrs, about 6 hrs, about 7 hrs, about 8 hrs, 1 hr, 2 hrs, 3 hrs, 4 hrs, 5 hrs, 6 hrs, 7 hrs, 8 hrs, between about 1 hr to about 3 hrs, between about 3 hrs to about 6 hrs, between about 4 hrs to about 8 hrs. In one embodiment, the blood treated with the alkylating substance neutralizing agent can be incubated for an additional 3 hrs at room temperature.

In some embodiments, upon the neutralization of the alkylating substance, the treated sample can be further processed. Further processing can include, but is not limited to, removing all/or some of the remaining cellular components, and/or removing leukocytes using a leukofiltration device and/or differential centrifugation.

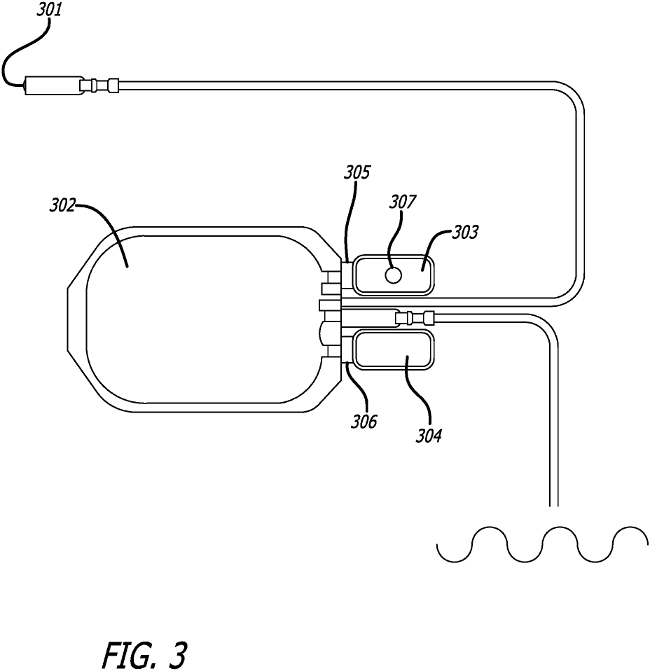

FIG. 3 depicts a sample collection and microbial contaminant inactivation system described herein. The systems can use a venipuncture apparatus to collect blood. In some embodiments, the venipuncture apparatus can be a phlebotomy needle, any type of needle, and/or any type of syringe. As depicted in FIG. 3, the venipuncture apparatus can be phlebotomy needle (301). Phlebotomy needle (301) can be in fluid contact with single sterile bag (302). Blood is collected by phlebotomy needle (301) and flows into single sterile bag (302). Single sterile bag (302) can include a buffered anticoagulant diluent. In some embodiments, the anticoagulant diluent is citrate dextrose phosphate (CPD) solution.

Sterile sample collection bag (302) can be configured to attach to one or more administration means. In some embodiments, sample collection bag (302) is attached to at least one or more administration means. Attachment occurs at positions (305) and or (306) FIG. 3 depicts first administration means (303) and second administration means (304), each configured to attach to the sample collection bag (302). First administration means (303) can include an alkylating substance contained in a separate container or capsule (307). Immediately prior to use, the alkylating agent container is ruptured allowing the alkylating agent to mix with a suitable buffer such as CPD prior to introduction into the sample collection bag (302). In some embodiments, the alkylating substance is ZD010. After introduction of the alkylating agent into the sample collection bag (302) containing the alkylation agent and sample the mixture is incubated for a suitable period (e.g. six hours). Next a neutralizer is aseptically introduced into the sample collection bag (302) through the second administration means (304). In some embodiments, the neutralizing agent is STS.

In some embodiments, a suitable incubation period can be from about 30 seconds (secs) to about 72 hrs, from about 20 min to about 24 hrs, or from about 60 min to about 8 hrs, and at temperatures from about 0 to about 100° C., from about 10 to about 60° C., or from about 20 to about 40° C.; and at a pH from about 1 to about 14, from about 4 to about 9, or from about 6 to about 8; and at concentrations from about 10 nM to about 10 mM, from about 1 µM to about 1 mM, or from about 100 µM to about 500 µM.

In other embodiments, a suitable incubation period can be from 30 secs to 72 hrs, from 20 min to 24 hrs, or from 60 min to 8 hrs, and at temperatures from 0 to 100° C., from 10 to 60° C., or from 20 to 40° C.; and at a pH from 1 to 14, from 4 to 9, or from 6 to 8; and at concentrations from 10 nM to 10 mM, from 1 µM to 1 mM, or from 100 µM to 500 µM.

In some embodiments, the incubation period with the addition of the neutralizer/neutralizing agent into the single sterile bag (302) can be from about 10 mins to about 10 hrs or more depending on the neutralizing agent being used. In other embodiments, the incubation period with the addition of the neutralizer/neutralizing agent into the single sterile bag (302) can be from 10 mins to 4 hrs, from 10 mins to 5 hrs, from 10 mins to 6 hrs, from 10 mins to 7 hrs, from 10 mins to 8 hrs, from 10 mins to 9 hrs, from 10 mins to 10 hrs, from 10 mins to 11 hrs, from 10 mins to 12 hrs, from 10 mins to 13 hrs, from 10 mins to 14 hrs, from 10 mins to 15 hrs, from 15 mins to 5 hrs, from 15 mins to 10 hrs, from 15 mins to 12 hrs, from 30 mins to 5 hrs, from 30 mins to 10 hrs, from 30 mins 12 hrs, from about 10 mins to about 4 hrs, from about 10 mins to about 5 hrs, from about 10 mins to about 6 hrs, from about 10 mins to about 7 hrs, from about 10 mins to about 8 hrs, from about 10 mins to about 9 hrs, from about 10 mins to about 10 hrs, from about 10 mins to about 11 hrs, from about 10 mins to about 12 hrs, from about 10 mins to about 13 hrs, from about 10 mins to about 14 hrs, from about 10 mins to about 15 hrs, from about 15 mins to about 5 hrs, from about 15 mins to about 10 hrs, from about 15 mins to about 12 hrs, from about 30 mins to about 5 hrs, from about 30 mins to about 10 hrs, or from about 30 mins about 12 hrs.

After the neutralization incubation period has lapsed the sample can be stored for later use or processed immediately. Leukocytes, red blood cells, and/or platelets can be removed if desired using the methods described herein as can the red blood cell components. Additionally, residual alkylating substance and/or neutralizing agent (including break down products of the reaction) can be removed as described herein, or using methods known to those of ordinary skill in the art.

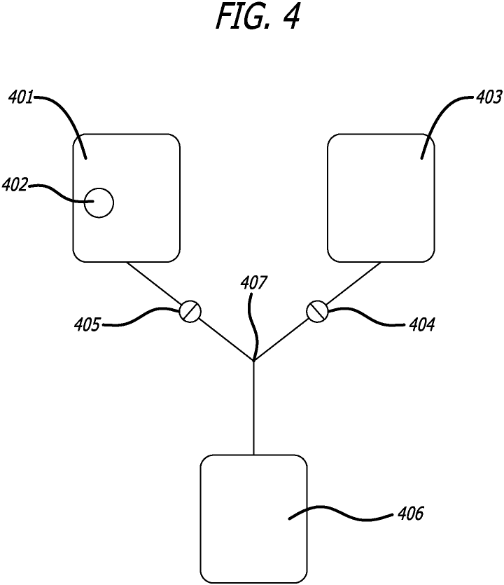

FIG. 4 depicts a multi-bag collection system. This system can comprise one or more collection bags/bags. In some embodiments, this system comprises 1 bag, 2 bags, 3 bags, 4 bags, 5 bags, 6 bags, 7 bags, 8 bags, 9 bags, 10 bags, at least 1 bag, at least 2 bags, at least 3 bags, at least 4 bags, at least 5 bags, at least 6 bags, at least 7 bags, at least 8 bags, at least 9 bags, at least 10 bags, 1 or more bags, 2 or more bags, 3 or more bags, 4 or more bags, 5 or more bags, 6 or more bags, 7 or more bags, 8 or more bags, 9 or more bags, or 10 or more bags.

FIG. 4 illustrates a multi-bag system comprising two, three or more bags, i.e. first bag (401) including CPD and having a ZD010-containing capsule disposed therein a second bag (403) including a neutralizing agent, such as but not limited to STS , and third bag (406). In one embodiment, this system is sterilely connected to an approved collection device (406). In another embodiment, the system is pre-attached to 406. In some embodiments, the multi-bag system further comprises one or more valves. The multi-bag system can comprise at least 1, at least 2, at least 3, at least 4, at least 5, at least 6, at least 7, at least 8, more than 1, more than 2, more than 3, more than 4, more than 5, more than 6, more than 7, or more than 8 valves. The valves can be a standard stopcock, pressure sensitive valve, or other means for establishing or restricting fluid communication between bags (401) and (403) with bag (406).

First bag (401) can comprise a buffered anticoagulant diluent. In some embodiments, the buffered anticoagulant diluent is aCPD solution. An alkylating substance can be sequestered within inner bag (402). Second bag (403) can include a neutralizing agent. Sample is collected in third bag (406). Third bag (406) can be attached to the system at point of attachment (407) at the time the sample is collected, or third bag (406) can be attached after the sample is collected and prior to alkylation agent treatment. The alkylating substance can be released from inner bag (402) using a suitable means such as external pressure on inner bag (402). The alkylating substance mixes with the CPD and is allowed to flow into collection bag (406) by valve (405). The sample is then incubated with the alkylating agent solution for a time sufficient to inactivate microbial contaminants. In some embodiments, a time sufficient to inactivate microbial contaminants can be from five minutes to 24 hours including all time intervals there between, from about 1 to about 12 hours, from about 2 to about 8 hours, or from about 4 to about6 hours

After inactivation of the microbial contaminants, valve (404) can be opened to allow the neutralizing substance to flow into third bag (406) where the alkylating agent treated sample is mixed with the neutralization agent.

After the neutralization incubation period has lapsed the sample can be stored for later use or processed immediately. Leukocytes, red blood cells, and/or platelets can be removed if desired using the methods described herein as can the red blood cell components. Additionally, residual alkylating substance and/or neutralizing agent (including break down products of the reaction) can be removed as described herein, or using methods known to those of ordinary skill in the art.

The following Examples set forth representative aspects of the methods disclosed herein. These examples are not limiting and persons of ordinary skill in the art would be familiar with means for modifying the following Examples or using other methods to achieve the desired results. Samples possessed according to these examples can be whole blood, blood products collected using apheresis blood or other liquid biological materials.

### EXAMPLES

### Example 1

### Synthesis of ZD010, N1,N4-bis(3-(Aziridin-1-yl)propyl)-N1,N4-dimethylbutane-1,4-Diamine

A. Synthesis of aziridine: 2-Chloroethylamine hydrochloride, 58.4 g (0.503 mol) was dissolved in 100 ml water. The solution was added dropwise with stirring to a solution of 56.4 g sodium hydroxide in 20 mL of water. After additional stirring for 2.5 h at 50° C., aziridine was purified by distillation under partial vacuum. Solid NaOH was added in portions to the distillate under vigorous stirring and cooling at temperature 0-8° C. The mixture was stirred at this temperature for 30 min. The liquid was decanted from the solid NaOH, and the top layer was separated to give 22.5 g of wet aziridine. This material was dried by addition of small portions of powdered KOH and decanting after each portion, until KOH retained dry appearance. The resulted dry aziridine stored under KOH pallets at -20° C. Yield, 16.02 g, 74% of clear liquid.

B. Synthesis of 2-(1-aziridinyl)propanal mono-methyl acetal, IV: Acrolein, 6.65 g, 7.93 ml, 0.120 mol was added to 100 ml MeOH. The solution was flushed with argon (Ar) and cooled under Ar in a dry ice bath. Aziridine, 4.99 g, 6.00 ml, 0.124 mol was added dropwise and on stirring. The dry ice bath was removed, and the reaction mixture was left to room temperature. Thus, obtained solution of 2-(1-aziridinyl)propanal mono-methyl acetal, IV was stored sealed under Ar and at 20° C. 1H NMR (300 MHz, CD3OD) δ: 4.66 (t, J = 5.54 Hz, 1H), 3.36 (s, 3H), 2.30-2.44 (m, 2H), 1.79-1.93 (m, 2H), 1.76-1.79 (m, 2H), 1.30-1.33 (m, 2H). 13C NMR (75 MHz, CD3OD) δ: 97.9, 57.5, 36.5, 26.6.

C. Synthesis of N1,N4-bis(3-(aziridin-1-yl)propyl)-N1,N4-dimethylbutane-1,4-diamine, VI: The methanol solution of compound IV from B was cooled in an ice bath. N,N′-Dimethylbutane-1 ,4-diamine, 5.85 g, 50.4 mmol was added dropwise and on stirring. The bath was removed, and after 30 min sodium borohydride, 10 g was added on portions on stirring and cooling at +4° C. After 4 hours at room temperature and aqueous work up and extraction with ether the product was purified by silica gel chromatography. The fractions containing the product were evaporated and the residue was subjected to vacuum distillation to give 3.84 g compound VI as a light-yellow oil. 1H NMR (300 MHz, C6D6) δ: 2.43 (t, J = 7.2 Hz, 4H), 2.30 (m, 4H), 2.13 (t+s, J = 6.7 Hz, 10H), 1.75 (m, 4H), 1.55 (m, 4H), 1.51 (m, 4H), 0.79 (m, 4H). 13C NMR (75 MHz, C6D6) δ: 60.74, 58.55, 56.49, 42.52, 28.77, 27.50, 26.11. MS (Electrospray, positive mode) m/z: 283.1, calc. [M+H]+ 283.2.

### Example 2

### Neutralization of Residual ZD010 by Sodium Thiosulfate

Sodium thiosulfate (Na₂S₂O₃) reacts quickly with ZD010′s aziridine groups opening the ring and converting it to a biologically well-tolerated thiosulfate ester, which are expected to be subject to fast renal excretion. The rate of reaction of ZD010, 100 µM with 1 mM Na₂S₂O₃ in PBS was determined by LCMS analysis of the reaction mixture. The reaction follows first-order kinetics with rate constants of 0.00614 min⁻¹ at 6° C., and 0.0379 min⁻¹ at 25° C. At this reaction rate, the half-live of compound VI in 10 mM Na₂S₂O₃ and 25° C. will be 1.83 min, which after 2 h will result in 5.5×10⁻¹⁹ M residual ZD010 concentration. LCMS analysis of the reaction product confirmed that it was the bis-thiosulfate ester having the following structure:

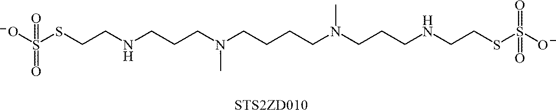

S,S′-(7,12-dimethyl-3,7,12,16-tetraazaoctadecane-1,18-diyl) bis(O-hydrogen sulfothioate) Sodium thiosulfate (and other neutralizing agents) can be used in solution or coupled to a chemically inert support as described further below.

### Example 3

### Sample Collection Using the Apparatus of FIG. 2

Step 1 - collection of blood by phlebotomy needle (201) into collection bag (202) including an anticoagulant solution, such as but not limited to, CPD solution. As used herein CPD will refer to any suitable anticoagulant solution known to those skilled in the art of transfusion hematology. In one embodiment, the CPD solution comprises citric acid monohydrate 0.327 grams per (g/) 100 mL, sodium citrate dihydrate 2.630 g/100 mL, monosodium phosphate monohydrate 0.222 g/ 100 mL, and dextrose (anhydrous) 4.640 g/ 100 mL (the diluent being purified sterile water). In step 2, approximated 10 mL of 10 mM ZD010 (203) is added to the CPD solution and incubated with the collected blood at room temperature (approximately 20° C.) for six hours. Step 3 provides a ZD010 solid phase removal column (204) up stream of a final treated sample collection bag. In some embodiments, an additional treatment can be performed using sodium thiosulfate (STS) (30 mM) (104) which can be added to the final treated sample collection bag (206). The STS treated blood can be incubated for an additional 3 hours at room temperature.

Once the ZD010 has been neutralized, the sample in Example 2 can be further processed. For example, the collected ZD010 treated sample can have some or all of the remaining cellular components removed. In one embodiment, leukocytes are removed using a leukofiltration device and/or differential centrifugation. Such techniques are well known to those of ordinary skill in the art.

Residual ZD010 and/or neutralizing agent (including break down products of the reaction) removal can be accomplished using a suitable solid phase system (103) that is insoluble in the treated media. A solid phase agent can be porous, microporous, macroporous, a gel type, or non-porous, and/or a combination thereof. In some embodiments, the solid phase agent can include high dispersity, and/or be a high surface area solid. The solid phase agent can be shaped as bead(s) or particle(s) of different size, e.g. ranging anywhere from about 1 µm to about 1 cm. The solid phase agent can chemically react with and covalently bind, or absorb, or otherwise sequester residual ZD010 and or the products of ZD010 neutralization and/or the neutralizing agent (including related breakdown products), followed by removal of the solid phase agent.

In some embodiments, the solid phase agent can be removed by filtration, sedimentation, or centrifugation, or a combination thereof. Alternatively, the treatment can be done by filtering of the media or composition through a cartridge including the solid phase agent, or by contact of the media or composition with the solid phase agent through a permeable or a semi-permeable membrane. In other embodiments, the treatment can be performed once (a single time), twice (two times), more than once, i.e. multiple times, or until the desired reduction of the compounds of neutralization or degradation of ZD010 is achieved, and which treatment can be done with a single solid phase agent, or with two or more different solid phase agents, either subsequently, or in a mixture.

In some aspects of the methods described herein, the solid phase agent is an activated carbon, a reversed-phase resin, a porous hydrophobic organic polymer, a microporous hydrophobic organic polymer. In some embodiments, the solid phase agent can be, but is not limited to, polystyrene resin, divinyl benzene cross-linked polystyrene resin, polyacrylate, or polymetacrylate resin modified with hydrophobic organic groups, such as C₄-C₁₈ alkyl groups.

In other embodiments, the solid phase agent is a polymer. The solid phase agent can be a cross-linked polymer. In some embodiments, the solid phase agent is a cross-linked polymer that can include thiosulfate groups ion-paired with acceptable cations, such as sodium and having the formula P—R—S—SO₃⁻Na⁺, where P is the polymer, R is a covalent bond or any divalent linker, and which groups react with the excess of the mercapto, or thiol type of neutralizing agent of formula R¹SH or R¹S⁻Cat⁺, where Cat⁺ is an acceptable cation, such as sodium by an exchange reaction resulting in covalent binding of the inactivator to the polymer through a disulfide bond as per the following formula, P—R—S—S—R¹, and release of thiosulfate anion, S₂O₃²⁻; or the said polymer can include epoxy or substitute epoxy attached to it, either directly or through a linker, and which epoxy groups react with the excess of the mercapto, or thiol type of neutralizing agent of formula R¹SH or R¹S⁻Cat⁺, where Cat⁺ is an acceptable cation, such as sodium, opening the epoxy groups and covalently attaching the neutralizing agent to the said polymer.

### Example 4

### Sample Collection Using the Apparatus of FIG. 3

The collection system depicted in FIG. 3 comprises a phlebotomy needle (301) in fluid contact with a single sterile bag (302) including CPD used to collect, process and optionally store the sample. Attached to the bag are administration means that allow for aseptic ZD010 introduction (303) followed by aseptic neutralizer (STS) addition (304) after a suitable ZD010 incubation period such as from 30 sec to 72 hours, from 20 min to 24 h, or from 60 min to 8 h, and at temperatures from 0 to 100° C., from 10 to 60° C., or from 20 to 40° C.; and at a pH from 1 to 14, from 4 to 9, or from 6 to 8; and at concentrations from 10 nM to 10 mM, from 1 µM to 1 mM, or from 100 µM to 500 µM. Neutralization agent incubation periods can range from 10 minutes to 10 hours or more depending on the neutralizing agent used. For STS, the reaction follows first-order kinetics with rate constants of 0.00614 min⁻¹ at 6° C., and 0.0379 min⁻¹ at 25° C. At this reaction rate, the half-live of compound VI in 10 mM Na₂S₂O₃ and 25° C. will be 1.83 min, which after 2 h will result in 5.5×10⁻¹⁹ M residual ZD010 concentration.

After the chosen neutralization period has lapsed the sample can be stored for later use or processed immediately. Leukocytes, red blood cells, and/or platelets can be removed if desired using the methods set forth above as can the red blood cell components. Additionally, residual ZD010 and/or neutralizing agent (including break down products of the reaction) can be removed as explained herein, or know to those skilled in the art.

### Example 5

### Sample Collection Using the Apparatus of FIG. 4

FIG. 4 depicts a multi-bag system. First collection bag (401) includes CPD solution and ZD010 sequestered within an inner bag or other suitable container (402). The ZD010 is released from inner bag (402) (or equivalent) using a suitable means such as external pressure on inner bag (402), and the ZD010 mixes with the CPD. Next third bag (406) which can be connected to the system at the time the sample is collected or attached subsequently having already been used to collect the sample is mixed with the ZD010 solution. Mixing is achieved by opening the valve (stopcock or other suitable valve means) and allowing the ZD010 solution to flow into third bag (406). The ZD010/sample mixture is then incubated for a time sufficient to inactivate microbial contaminants. In some embodiments, a time sufficient to inactive microbial contaminants includes 5 minutes to 24 hours including all time intervals there between. In one embodiment, the incubation period is six hours.

Next s valve (404) is opened allowing the ZD010 the STS stored in bag (403) to flow into third bag (406), where the ZD010 treated sample and neutralization agent are mixed.

After the chosen neutralization period has lapsed, the sample can be stored for later use or processed immediately. Leukocytes, red blood cells, and/or platelets can be removed if desired using the methods set forth herein as can the red blood cell components. Additionally, residual ZD010 and/or neutralizing agent (including breakdown products of the reaction) can be removed as explained herein, or known to a person of ordinary skill in the art.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A method for inactivation or reduction of microbial contaminants in a sample, comprising:
(i) treating the sample with an inactivating compound having Structure I: 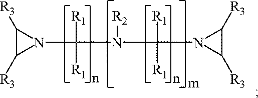
(ii) incubating the treated sample for a time sufficient to inactivate or reduce at least one microbial contaminant in the sample;
(iii) adding one or more neutralizing agents to the treated sample following said incubation step; and
(iv) incubating said treated sample in the presence of said one or more neutralizing agents for a time sufficient to eliminate or reduce toxicity of the inactivating compound.

2. A method for inactivation or reduction of microbial contaminants in a sample, comprising:
(i) treating the sample with an inactivating compound comprising ZD010: 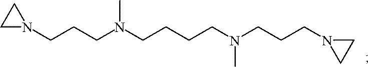
(ii) incubating the treated sample for a time sufficient to inactivate or reduce at least one microbial contaminant in the sample;
(iii) adding one or more neutralizing agents to the treated sample following said incubation step; and
(iv) incubating said treated sample in the presence of said one or more neutralizing agents for a time sufficient to eliminate or reduce toxicity or other undesirable properties of ZD010.

3. The method according to claims 1 or 2, wherein the one or more neutralizing agents are nucleophilic compounds which eliminate the alkylating properties of the inactivating compound by reacting with and opening of the aziridine rings of the inactivating compound.

4. The method of claim 3, wherein the one or more neutralizing agents is a thiosulfate, wherein the thiosulfate is sodium thiosulfate.

5. The method according to claims 1 or 2, wherein the one or more neutralizing agents is covalently bound to a solid phase support.

6. The method according to claims 1 or 2, further comprising removal of residual inactivating compound, neutralization products and/or their residual by-products.

7. The method of claim 6, wherein a solid phase agent absorbs the residual inactivating compound, neutralization products and/or their residual by-products.

8. The method according to claims 1 or 2, wherein the microbial contaminants are infectious disease causing organisms, viruses, including enveloped and non-enveloped viruses, DNA or RNA viruses and bacteriophages, prokaryote, bacteria, including Gram-positive or Gram-negative bacteria, spore forming bacteria or bacterial spores, or mycoplasma or any combination thereof.

9. The method according to claims 1 or 2, wherein the sample comprises whole blood.

10. A method for inactivation or reduction of microbial contaminants in a sample, comprising:
(i) treating a sample comprising whole blood with an inactivating compound comprising ZD010; 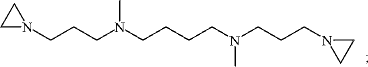
(ii) incubating the treated sample for a time sufficient to inactivate or reduce at least one microbial contaminant in the sample selected from the group comprising infectious disease causing organisms, viruses, including enveloped and non-enveloped viruses, DNA or RNA viruses and bacteriophages, prokaryote, bacteria, including Gram-positive or Gram-negative bacteria, spore forming bacteria or bacterial spores, or mycoplasma and any combination thereof;
(iii) adding sodium thiosulfate to the treated sample following said incubation step; and
(iv) incubating said treated sample in the presence of sodium thiosulfate for a time sufficient to eliminate or reduce toxicity or other undesirable properties of ZD010.

11. A sample collection and microbial contaminant inactivation system comprising:
a venipuncture apparatus in fluid contact with a sample collection bag having a buffered anticoagulant diluent therein;
an administration means attached to said sample collection bag for aseptically introducing an alkylating substance into said bag;
a second means attached to said sample collection bag for introducing an alkylating substance neutralizing agent into said sample collection bag;
and an administration means attached to said sample collection bag for aseptically removing said sample from said sample collection bag.

12. The system of claim 11, wherein said buffered anticoagulant diluent is citrate dextrose phosphate.

13. The system of claim 11, wherein said alkylating substance is ZD010.

14. The system of claim 13, wherein said alkylating substance neutralizing agent is sodium thiosulfate.

15. The system of claim 14, further comprising a leukocyte reduction apparatus.

16. The system of claim 14, wherein said sample is whole blood.

17. A sample collection and microbial contaminant inactivation system comprising:
a venipuncture apparatus in fluid contact with a first sample collection bag having a buffered anticoagulant diluent therein;
a device attached to said first sample collection bag for aseptically introducing an alkylating substance into said first sample collection bag;
a means for removing said alkylating substance from said sample wherein said means for removing said alkylating substance from said sample is separate from and in fluid contact and downstream from said first sample collection bag;
a second sample collection bag aseptically attached to said administration means for removing said alkylating substance from said sample;
an administration means attached to said second sample collection bag for introducing a residual alkylating substance neutralizing agent into said second sample collection bag;
and an administration means attached to second sample collection bag for aseptically removing said sample from said sample collection bag.

18. The system of claim 17, wherein said buffered anticoagulant diluent is citrate dextrose phosphate.

19. The system of claim 17, wherein said alkylating substance is ZD010.

20. The system of claim 17, wherein said alkylating substance neutralizing agent is sodium thiosulfate.

21. The system of claim 17, wherein said means for removing said alkylating substance from said sample comprises a chromatography substrate housed in a column or other suitable flow through device.

22. The system of claim 17, further comprising a leukocyte reduction apparatus.

23. The system of claim 17, wherein said sample is whole blood.

24. A sample collection and microbial contaminant inactivation system comprising:
an outer bag having a buffered anticoagulant diluent therein;
an inner bag included within said outer bag, wherein said inner bag includes an alkylating substance for introduction into said outer bag.

25. The system of claim 24, wherein said buffered anticoagulant diluent is citrate dextrose phosphate.

26. The system of claim 24, wherein said alkylating substance is ZD010.

27. The system of claim 24, wherein said alkylating substance neutralizing agent is sodium thiosulfate.

28. The system of claim 24, wherein said means for removing said alkylating substance from said sample comprises a chromatography substrate housed in a column or other suitable flow through device.

29. The system of claim 24, further comprising a leukocyte reduction apparatus.

30. The system of claim 24, wherein said sample is whole blood.
